(19) United States
(12) Patent Application Publication
(10) Pub. No.: US 2021/0289757 A1
(43) Pub. Date: Sep. 23, 2021

(54) DORSAL AORTIC INJECTION METHOD OF CHICKEN EMBRYOS FOR IMPROVING TRANSGENIC EFFICIENCY

(71) Applicant: GUANGXI UNIVERSITY, Nanning (CN)

(72) Inventors: Xibang ZHENG, Nanning (CN); Gonghe LI, Nanning (CN); Fei LI, Nanning (CN); Jinyu WEI, Nanning (CN); Longqin WEI, Nanning (CN); Xiaoming XU, Nanning (CN); Qingbo XING, Nanning (CN); Lingkang LIU, Nanning (CN); Zonghai WEI, Nanning (CN); Lisui SU, Nanning (CN); Yanfei TANG, Nanning (CN); Junjiang QIN, Nanning (CN); Juxiang LIU, Nanning (CN); Wenlue HUANG, Nanning (CN); Pengxia WANG, Nanning (CN); Yanyu XIAO, Nanning (CN); Mengyuan XU, Nanning (CN)

(21) Appl. No.: 17/128,449

(22) Filed: Dec. 21, 2020

(30) Foreign Application Priority Data
Mar. 19, 2020 (CN) 202010196673.4

(51) Int. Cl. A01K 45/00; G01N 21/3563; G01N 33/08

(52) U.S. Cl. CPC A01K 45/007; G01N 2021/3155; G01N 33/085; G01N 21/3563

(57) ABSTRACT

A dorsal aortic injection method of chicken embryos for improving transgenic efficiency, comprising the following steps: injecting adeno-associated virus particles into the dorsal aorta of each 2.5-day-old chicken embryo outside eggshell, individually transferring the injected chicken embryos into their recipient eggshells (coming from double yolk eggs), and proceeding to hatch. The disclosure realizes the outside-eggshell injection of the chicken embryo dorsal aorta through the modified dorsal aortic injection method, improves the accuracy of injection, and also realizes the direct observation of the development process of the chicken embryos. The survival rate of embryos, hatch rate and the efficiency of transgene expression in the modified dorsal aortic injection group are significantly higher than those in the subgerminal cavity injection group and the classic dorsal aortic injection group, indicating that the modified method improves the hatch rate of transgenic chicken embryos and EGFP expression efficiency.

### CROSS REFERENCE TO RELATED APPLICATION

This application claims the priority of Chinese Patent Application NO. 202010196673.4, entitled “Dorsal aortic injection method of chicken embryos for improving transgenic efficiency” filed with China National Intellectual Property Administration on Mar. 19, 2020, which is incorporated herein by reference in its entirety.

### TECHNICAL FIELD

The disclosure relates to the field of transgenic biotechnology, in particular to a dorsal aortic injection method of chicken embryos for improving transgenic efficiency.

### BACKGROUND ART

Since the 1980s, transgenic poultry has been one of the hot spots in the field of biotechnology, which has important application value in improving the rate of meat and egg production of poultry, improving disease resistance and building bioreactor model. Compared with the bioreactor of mammalian, the bioreactor of poultry oviduct has many advantages, such as shorter incubation time (3 weeks on average), shorter generation interval (20 weeks on average), faster reproductive speed, lower cost and higher yield.

The production of transgenic chickens is technically challenging, because the eggs of chickens are tightly connected to the yolk, so it is impossible to culture embryos in vitro to produce transgenic chickens through nuclear transfer technology like mammals. Until 1988, Perry developed the chicken embryo culture system (System I-III). At present, System II and System III surrogate eggshell culture methods are commonly used. In the System II, the whole donor embryos from freshly fertilized eggs are respectively transferred to an eggshell of recipient (about 3-5 g heavier than the donor); in the System III, the chicken embryos cultured for 2.5 days are individually transferred to a larger eggshell of recipient (30-40 g heavier than the donor), and proceed to hatch. Although subgerminal cavity injection is simple, the chicken embryos need two rounds of surrogate eggshell culture after injection, which causes mechanical damage to them, and their survival rate is low.

Classic dorsal aortic injection is to inject foreign genes into the dorsal aorta of HH14-16 chicken embryos after opening a window at the blunt end of eggshell, and finally seal the window and proceed to hatch. The advantage of the classic dorsal aortic injection is that surrogate eggshell culture is not needed, while the disadvantage is that the accuracy of inside-eggshell injection is low, and the efficiency of transgene is also decreased. Therefore, the classic dorsal aortic injection needs to be improved.

### SUMMARY OF THE INVENTION

The purpose of the disclosure is to provide a dorsal aortic injection method of chicken embryos for improving transgenic efficiency in order to solve the existing problems.

In order to achieve the above purpose, the present disclosure provides a dorsal aortic injection method of chicken embryos for improving transgenic efficiency, comprising the following steps:

a. selecting lately laid fertilized eggs as donors, and incubating them for 2.5 days at 37.8° C. and 60% relative humidity in an incubator;

b. slowly pouring the surviving embryo from each fertilized egg into a weighing disk;

c. injecting virus particles into the dorsal aorta of each embryo;

d. selecting double yolk eggs as recipient eggs, disinfecting the recipient eggs, opening a window at the blunt end, and discarding the contents;

e. individually transferring the surviving embryos to their recipient eggshells after the injection, sealing the window with parafilm and setting the window upward, and placing the embryos in the incubator, proceeding to hatch.

In one embodiment, the disinfecting comprising the following steps: cleaning the outer surface of fertilized eggs and the recipient ones with 0.01% benzalkonium bromide solution, and spraying 75% alcohol for disinfection.

In one embodiment, the method for opening a window on the recipient eggs comprising the following step: opening a window with a diameter of 40 mm at the blunt end of the recipient eggs.

In one embodiment, the dorsal aortic injection method comprising the following step: injecting 2 μL of virus particles into the dorsal aorta of a chicken embryo through a microinjection needle under a stereo microscope with the aid of a fiber optic illuminator.

In one embodiment, Handi-Wrap film is selected as the parafilm.

In one embodiment, the fertilized eggs and the recipient eggs are both from the same species.

In one embodiment, the double yolk eggs are selected as recipients which are 30-40 g heavier than the donors.

Compared with the prior art, the present disclosure has the following beneficial effects:

The present disclosure realizes the outside-eggshell injection of chicken embryos through the modified dorsal aortic injection method, and improves the accuracy of injection. The injected chicken embryos are individually transferred to their surrogate eggshells (from double yolk eggs) for culturing until to hatch. It also realizes the direct observation of the development process of chicken embryos. The survival rate of embryos, hatch rate, and efficiency of transgene expression in the modified dorsal aortic injection group are significantly higher than those in the subgerminal cavity injection group and the classic dorsal aortic injection group, indicating that the modified dorsal aortic injection method improves the hatch rate and EGFP expression efficiency of transgenic chicken embryos.

### BRIEF DESCRIPTION OF THE DRAWINGS

FIG. 1 is a photograph showing the operation process of the modified dorsal aortic injection and surrogate eggshell culture according to the present disclosure;

FIG. 2 is a histogram showing the effects of different injection methods on the survival rate of chicken embryos according to the present disclosure;

FIG. 3 is a histogram showing the effects of different injection methods on the hatch rate of chicken embryos according to the present disclosure;

FIG. 4 is a histogram showing the effects of different injection methods on the EGFP expression efficiency in chicken embryos according to the present disclosure.

### DETAILED DESCRIPTION OF THE EMBODIMENTS

The technical scheme of the present disclosure will be clearly and completely described below combined with the drawings in the examples of the present disclosure. Obviously, the described example is only a part of the present disclosure, rather than all the examples.

The fertilized eggs and infertile ones (recipients in system II) used in this experiment were purchased from Guangxi Nanning Fufeng Agriculture and Animal Husbandry Co., Ltd., and the double yolk eggs (recipients in system III) were purchased from Nanning Liangfeng Agriculture and Animal Husbandry Co., Ltd., AAV virus particles (HBAAV-CMV-EGFP) for injection were purchased from Hanbio Biotechnology Co., Ltd., with a titer of 1×10⁸ TU/ML, TU (Transducing Units).

The experimental groups were as follows: the subgerminal cavity injection of the X-stage chicken embryos group (hereinafter is subgerminal cavity injection group in short), the dorsal aortic injection of the HH14-16 stage chicken embryo group (hereinafter is classic dorsal aortic injection group in short), and the modified dorsal aortic injection of the HH14-16 stage chicken embryos group (hereinafter is modified dorsal aortic injection group in short). For each experimental group, 30 of fertilized eggs were injected each time, and 5 independent experiments were performed. A total of 150 fertilized eggs were injected in each group. The blank control group did not undergo any treatment, 12 of fertilized eggs were incubated each time, and a total of 60 fertilized eggs were incubated.

During the incubating period, the development of the chicken embryos was observed and the survival rate of the chicken embryos was recorded. The hatch rate was calculated. For freshly dead chicks or chicks being sacrificed to death, the expression of enhanced green fluorescent protein (EGFP) on the surface of body and splanchnic tissues from the chickens was observed by fluorescent protein flashlight, and the EGFP expression efficiency was calculated.

Subgerminal cavity injection: lately laid fertilized eggs were selected as donors, infertile ones that 3-5 g heavier than the donors were selected as recipients in System II. The donors and the recipients were weighed and paired one by one. The fertilized eggs and the recipient ones were washed with 0.01% benzalkonium bromide solution, and 75% alcohol was sprayed for disinfection. A 32 mm diameter window was cut at the pointed end of each recipient egg with a micro electric grinder, the contents were discarded, and the inner and outer surfaces were rinsed with distilled water. With the window facing down, the recipient eggs were placed in a ceramic dish lined with moist gauze. The donor eggs were gently tapped to crack the shells in a vertical flow clean bench, the whole chicken embryos were individually moved into their recipient eggshells in System II, the blastoderms were floated. 2 μL of EGFP-AAV virus solution was aspirated into a microinjection needle thorough a mouth pipette, then injected into the subgerminal cavity of each embryo under a stereo microscope with the aid of a fiber optic illuminator. Saran Wrap film (5 cm×5 cm) was used to seal the window on the eggshell, and the chicken embryos were put in an incubator at 37.8° C. and 60% relative humidity for 2.5 days with the window facing down. For surviving chicken embryos, double yolk eggs which are 30-40 g heavier than the donors were selected as recipients in System III. The preparation of recipient eggshells was the same as System II, except that the window was opened at the blunt end of the recipient eggs with a diameter of 40 mm. The surviving chicken embryos were respectively transferred to their recipient eggshells in System III, the window was sealed with Handi-Wrap film, and continued to culture until hatch with the window facing upward.

Classic dorsal aortic injection: The lately laid fertilized eggs were placed in the incubator to culture for 2.5 days, the blunt end of the fertilized eggs was put upward and horizontally for 1-2 hours. The eggs were candled to determine the location of the air chamber, and marked with a pencil. The eggshells were wiped with 75% alcohol. Forceps were used to open a window with a diameter of 1-1.5 cm in the center of the marked area. A little amount of sterile saline was added on the surface of the shell membrane. The membrane was torn, and 2 μL of EGFP-AAV virus solution was aspirated into a microinjection needle thorough a mouth pipette, then injected into the dorsal aorta of each embryo under a stereo microscope with the aid of a fiber optic illuminator. Parafilm was used to seal the window. With the window facing up, the injected embryos were proceeded to culture until hatch.

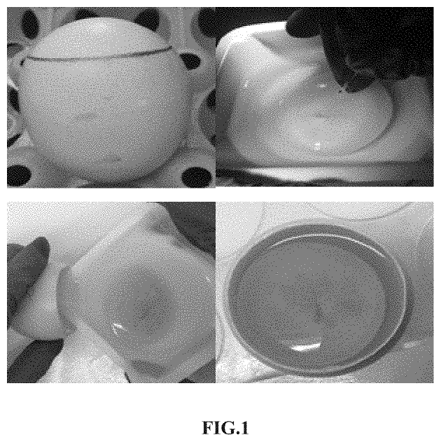

As shown in FIG. 1 that was a photograph showing the operation process of the modified dorsal aortic injection and surrogate eggshell culture; the lately laid fertilized eggs were used as donors, and their weight was recorded. The donor eggs were set in an incubator at 37.8° C. and 60% relative humidity for about 2.5 days, and the chicken embryos at HH14-16 stage were obtained. The double yolk eggs that 30-40 g heavier than the donors were selected as the recipients. The preparation of the recipient eggshells was the same as System III in subgerminal cavity injection. The surface of the donor eggs was wiped with 75% alcohol. The donor eggs were gently tapped to crack the shells in a vertical flow clean bench, and the surviving embryos were individually poured into a disposable weighing disk with the embryo facing up. 2 μL of EGFP-AAV virus solution was aspirated into a microinjection needle thorough a mouth pipette, and injected into the dorsal aorta of each embryo under a stereo microscope with the aid of a fiber optic illuminator. The injected chicken embryos were respectively transferred into their recipient eggshells, the window was sealed with Handi-Wrap film. With the window facing upward, the embryos proceeded to culture until hatch.

The survival rate of transgenic chicken embryos from day 8, 14, 18, 21 and the EGFP positive rate from day 14 to hatch was calculated. The one-way analysis of variance method of SPSS22.0 software was used to analyze the significant differences between the different experimental groups. P<0.05, indicating significant difference; P<0.01, indicating extremely significant.

Result

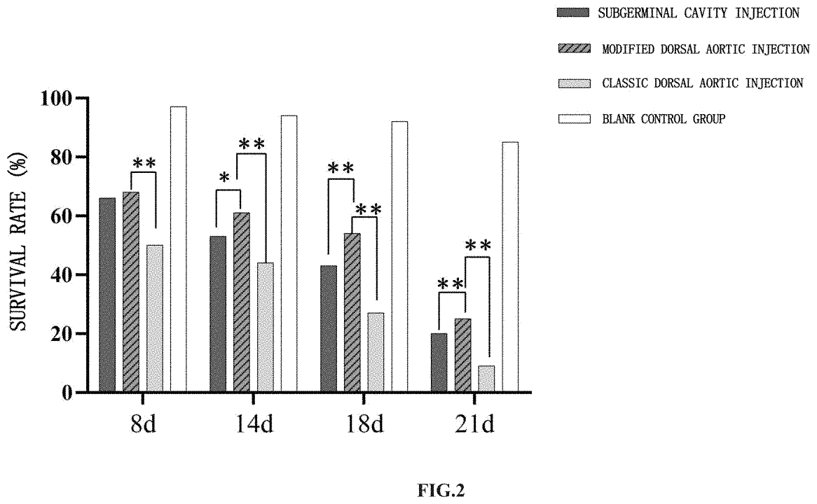

As shown in FIG. 2 and Table 1, when chicken embryos develop to 8 days, there is no significant difference in the survival rate of chicken embryos between the subgerminal cavity injection group and the classic dorsal aortic injection group (P>0.05). However, the survival rate of chicken embryos in the modified dorsal aortic injection group is higher than that in the classic dorsal aortic injection group and the difference is significant (P<0.01). Over time, the survival rate of chicken embryos shows a downward trend. However, when the chicken embryos developed to 14, 18 and 21 days, the survival rate of embryos in the modified dorsal aortic injection group is significantly higher than that in the subgerminal cavity injection group (P<0.05 or P<0.01) and the classic dorsal aortic injection group (P<0.01).

TABLE 1
The effects of three injection methods on the survival rate of chicken embryos

| | | Survival rate (%) | | | |
|---|---|---|---|---|---|
| Groups | Injection methods | Day 8 | Day 14 | Day 18 | Day 21 |
| 1 | Subgerminal cavity injection group | 66 | 53 | 43 | 20 |
| 3 | Modified dorsal aortic injection group | 68 | 61 | 54 | 25 |
| 2 | Classic dorsal aortic injection group | 50 | 44 | 27 | 9 |
| 4 | Blank control group | 97 | 94 | 92 | 85 |

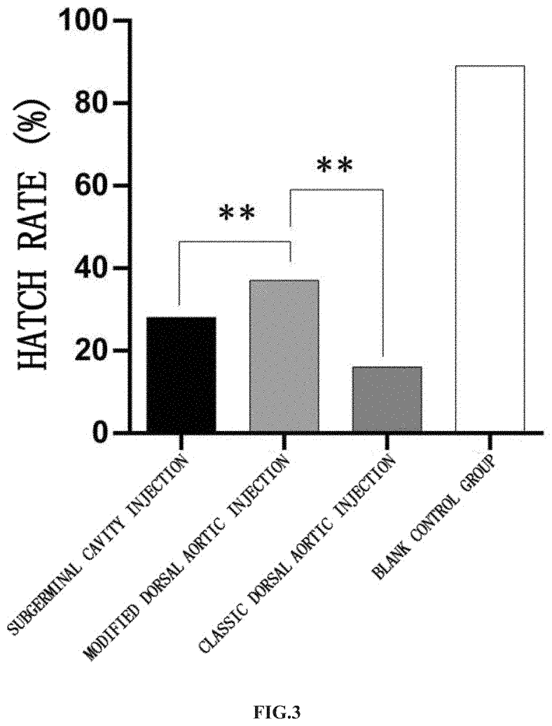

As shown in FIG. 3 and Table 2, the hatch rate of transgenic chicken embryos in the modified dorsal aortic injection group (37%) is significantly higher than that in the subgerminal cavity injection group (26%) and the classic dorsal aortic injection group (16%) (P<0.01).

TABLE 2
The effects of three injection methods on the hatch rate of chicken embryos

| Injection methods | Hatch rate (%) |
|---|---|
| Subgerminal cavity injection group | 28 |
| Modified dorsal aortic injection group | 37 |
| Classic dorsal aortic injection group | 16 |
| Blank control group | 89 |

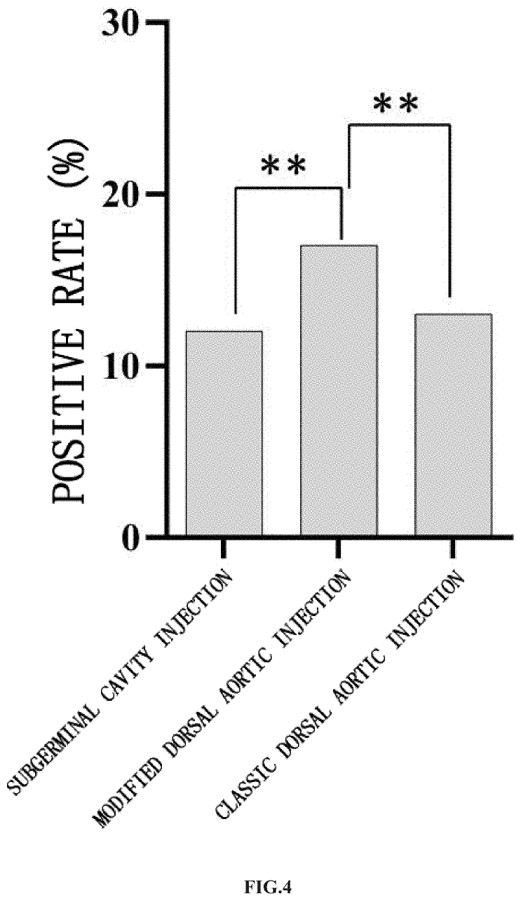

For freshly dead chicks or chicks being sacrificed to death, the fluorescent protein flashlight and its matching filter were used to observe the expression of green fluorescent protein. The results show that EGFP is mostly expressed in the heart, gallbladder, sternum, skin, beak and other parts; The GFP positive rate of chicken embryos in the modified dorsal aortic injection group is significantly higher than that in the subgerminal cavity injection group and the classic dorsal aortic injection group (FIG. 4, Table 3) (P<0.01).

TABLE 3
The effects of three injection methods on EGFP
expression efficiency in chicken embryos

| Injection methods | EGFP positive rate (%) |
|---|---|
| Subgerminal cavity injection group | 12 |
| Modified dorsal aortic injection group | 17 |
| Classic dorsal aortic injection group | 13 |

In this experiment, damage to embryos caused by the operation of surrogate eggshell culture may be one of the reasons for the gradual decline in survival rate of the embryos in System II and System III. In addition, the subgerminal cavity injection and dorsal aortic injection also cause mechanical damage to the embryos, which is also responsible for embryonic death. In this experiment, the hatch rate of transgenic chicken embryos in the modified dorsal aortic injection group is higher than that in the subgerminal cavity injection group, because the former undergoes only one round of surrogate eggshell culture, and the mechanical damage to the chicken embryos is less than the latter.

The microinjection at different development stages may directly affect the survival rate of embryos and the expression efficiency of foreign genes. The primordial germ cells (PGCs), as the precursor cells of sperm and eggs, are considered to be ideal target cells for preparing transgenic chickens. It is possible to obtain transgenic chickens by transfection or transduction of PGCs in vivo. The lately laid fertilized eggs have developed to the HH 10 stage, and there are 50,000-60,000 embryonic cells in the subgerminal cavity between the blastoderm and the yolk, of which only 40 are PGCs; at HH 14-16 stage (about 2.5 days of age), blood islands and vascular are formed, PGCs enter the blood circulation, and the number of PGCs increases to 100-200. This is also the reason why the EGFP positive rate in the modified dorsal aortic injection group is higher than that in the subgerminal cavity injection group. Compared with the classic dorsal aortic injection, the modified method realizes the injection outside eggshell, improves the accuracy of the injection, and further improves the EGFP positive rate in the embryonic tissue of transgenic chickens.

The genetic distance between species (the interval between species) is another reason that affects the hatch rate. As recipient egg whites, the thin egg whites were respectively isolated from the eggs of chicken, turkey, guinea fowl and duck, and chicken eggshells are used as the common recipients in System III. The hatch rates of chicken embryos in these different recipient egg whites and the same recipient eggshells were respectively 60.4%, 55.3%, 47.9% and 19.1%, which proves that the hatch rate of chicken embryos both in egg whites and eggshells of chicken is the highest. Therefore, the mortality of chicken embryos may be reduced by selecting the recipient eggs from the spices who have close genetic relationship with their donors during surrogate eggshell culturing. During the System II and System III in this experiment, both egg white and egg shells used as recipients were all from chicken, which avoids the above-mentioned risks and reduces the mortality of chicken embryos.

Avian embryos are highly dependent on eggshells during their development. Eggshells not only protect the embryos from physical stress and microbial infection, but also regulate water and provide minerals. The thickness of the recipient eggshells and the material of the parafilm affect the hatch rate. Improving oxygen supply through recipient eggshells is an important factor in embryo culture in vitro. Different recipient eggshells will bring about different hatch rates. In this experiment, double yolk eggs were used as the recipients in System III both from the modified dorsal aortic injection group and the subgerminal cavity injection group. Despite the mechanical damage caused by the microinjection, the hatch rate of transgenic chicken embryos in the modified dorsal aortic injection group reaches 37%. In the System III, when the recipient window is sealed by Handi-Wrap film, the hatch rate increased by 30% than when covered by Saran Wrap film.

All in all, the modified dorsal aortic injection realizes the dorsal aortic injection of the chicken embryos outside eggshell and direct observation of the development process of the chicken embryos, which improves the accuracy of injection, thereby increasing the hatch rate and the transgene expression efficiency of the transgenic chicken embryos.

In this experiment, the combination of dorsal aortic injection outside-eggshell and surrogate eggshell culture in System III is used to modify the classic dorsal aortic injection method, and further to improve the accuracy of injection, and increases the survival rate, hatch rate and transgene expression efficiency of transgenic chicken embryos.

The above are only preferred specific embodiments of the present disclosure, but the present disclosure is not limited to this. Anyone familiar with the technical field within the technical scope disclosed by the present disclosure, equivalent replacements or changes according to the technical solution and the inventive concept of the present disclosure shall be covered by the protection scope of the present disclosure.

What is claimed is:

1. A dorsal aortic injection method of chicken embryos for improving transgenic efficiency, comprising the following steps:
a. selecting lately laid fertilized eggs as donors and incubating them for 2.5 days at 37.8° C. and 60% relative humidity in an incubator;
b. slowly pouring the surviving embryo from each fertilized egg into a weighing disk;
c. injecting virus particles into the dorsal aorta of each embryo;
d. selecting double yolk eggs as recipient eggs, disinfecting the recipient eggs, opening a window at the blunt end, and discarding the contents;
e. individually transferring the whole chicken embryos to their recipient eggshells after the injection, sealing the window with parafilm and setting the window upward, and placing the whole embryos in the incubator, proceeding to hatch.

2. The dorsal aortic injection method of chicken embryos for improving transgenic efficiency according to claim 1, wherein the disinfecting comprising the following steps: cleaning the outer surface of fertilized eggs and the recipient ones with 0.01% benzalkonium bromide solution, and spraying 75% alcohol for disinfection.

3. The dorsal aortic injection method of chicken embryos for improving transgenic efficiency according to claim 1, wherein the method for opening a window on the recipient eggs comprising the following step: opening a window with a diameter of 40 mm at the blunt end of the recipient eggs.

4. The dorsal aortic injection method of chicken embryos for improving transgenic efficiency according to claim 1, wherein the dorsal aortic injection method comprising the following step: injecting 2 μL of virus particles into the dorsal aorta of each chicken embryo through a microinjection needle under a stereo microscope with the aid of a fiber optic illuminator.

5. The dorsal aortic injection method of chicken embryos for improving transgenic efficiency according to claim 1, wherein Handi-Wrap film is selected as the parafilm.

6. The dorsal aortic injection method of chicken embryos for improving transgenic efficiency according to claim 1, wherein the fertilized eggs and the recipient eggs are both from the same species.

7. The dorsal aortic injection method of chicken embryos for improving transgenic efficiency according to claim 1, wherein double yolk eggs are selected as recipient eggs which are 30-40 g heavier than the donor ones.
